(19) United States
(12) Patent Application Publication
(10) Pub. No.: US 2009/0123987 A1
(43) Pub. Date: May 14, 2009

(54) Extracting and purifying limit dextrinases

(76) Inventors: Michael Patane, Seaforth (AU); Mitchell Parker, Seaforth (AU); Akshat Talwalkar, Seaforth (AU)

Correspondence Address: Ballard Spahr Andrews & Ingersoll, LLP, SUITE 1000, 999 PEACHTREE STREET, ATLANTA, GA, 30309-3915, US

(21) Appl. No.: 11/991,059

(22) PCT Filed: Aug. 25, 2006
(86) PCT No.: PCT/AU2006/001240

§ 371 Date: Feb. 26, 2008

(30) Foreign Application Priority Data
Aug. 26, 2005 (AU) 2005904663

(51) Int. Cl. C12N 9/14

(52) U.S. Cl. 435/195

(57) ABSTRACT

The invention relates to improved methods for extracting and purifying limit dextrinase enzymes from cells, in particular plant cells. The process of the invention includes heating a cell homogenate at 4O° C., in the presence of divalent cations thus increasing the specific activity of the limit dextrinase.

### FIELD OF THE INVENTION

The invention relates to extracting and purifying an enzyme from a cell, particularly, but not exclusively, to extracting and purifying a limit dextrinase.

### BACKGROUND OF THE INVENTION

Limit dextrinase (EC 3.2.1.142), otherwise known as dextrin α-1,6-glucanohydrolase; R-enzyme; or amylopectin-1-6 glucosidase; is an enzyme that catalyses the hydrolysis of (1→6)-α-D-glycosidic linkages in α- and β-limit dextrins of amylopectin and pullulan.

Limit dextrinases have little or no activity on glycogen, incomplete action on amylopectin and complete action on a-limit dextrins. These enzymes release maltose from an -(1→6)-linkage and hence are particularly important in food industries for providing maltose.

The processes for obtaining commercial quantities of limit dextrinases tend to be difficult to operate on a commercial scale, in terms of requiring sophisticated fermentation technology, extraction and separation techniques, multiple steps and expensive reagents and equipment. Some processes are characterised by an unacceptable loss or wastage of limit dextrinases. Other processes tend to produce a non purified final product that has a sub-optimal specific activity.

In view of the above, there is a need for improved processes for purification of limit dextrinases.

### SUMMARY OF THE INVENTION

The invention seeks to at least minimise one or more of the above identified problems or limitations and/or to provide an improved process for purification of limit dextrinase.

In one aspect, the invention provides a process for purifying a limit dextrinase from a cell. The process includes a step of heating an extract of a cell formed from a solution including at least one divalent cation, to increase the specific activity of a limit dextrinase in the extract.

In another aspect, the invention provides a process for purifying limit dextrinase from a barley cell. The process includes the following steps:

(a) releasing limit dextrinase from a barley cell into a solution including calcium and magnesium to form an extract;

(b) heating the extract to increase the specific activity of limit dextrinase in the extract.

In another aspect, the invention provides a process for purifying limit dextrinase from a barley cell. The process includes the following steps:

(a) releasing limit dextrinase from a barley cell into a solution including calcium and magnesium to form an extract;

(b) heating the extract to increase the specific activity of limit dextrinase in the extract; and

(c) utilising anion exchange chromatography to purify limit dextrinase from the heated extract.

Typically the cell is a barley cell, such as a cell derived from a barley rootlet or grain.

In another aspect, the invention provides limit dextrinase produced by the process of the invention.

In another aspect, the invention provides a cell including limit dextrinase produced by the process of the invention.

### DETAILED DESCRIPTION OF THE EMBODIMENTS

As described herein, it has been found that creating a suitable buffered environment during extraction and conducting heat treatment of an extract of a barley cell or rootlet in a solution comprising at least one divalent cation permits the specific activity of the extract with respect to limit dextranase contained within it to be increased. For example, the specific activity of a heat treated extract of a barley rootlet formed from a example, the specific activity of a heat treated extract of a barley rootlet formed from a solution comprising 50 mM Calcium Chloride and 50 mM Magnesium Chloride was observed to increase 2.4 fold over a non heat treated sample (756.54 μmoles/min/mL compared with 312.09 μmoles/min/mL). Further, a heat treated extract containing 50 mM Calcium Chloride and 50 mM Magnesium Chloride was observed to have an improved specific activity (385.7 μmoles/min/mL) compared with a heat treated extract containing no Calcium and Magnesium (75.0 μmoles/min/mL).

This is a significant finding because it permits heat treatment, a purification step that is relatively simple to operate on a commercial scale, to be implemented with minimal loss of activity of limit dextrinase.

Thus in certain embodiments there is provided a process for purifying a limit dextrinase from a cell including the step of heating an extract of a cell formed from a solution including at least one divalent cation, to increase the specific activity of a limit dextrinase in the extract.

In other embodiments there is provided a process for increasing the specific activity of a limit dextrinase in a cell extract, said extract being one formed from a solution including at least one divalent cation. The process includes the step of heating the cell extract to increase the specific activity of a limit dextrinase in the extract.

It is believed that the specific activity of the extract is increased because the divalent cation protects limit dextrinase from denaturation at temperatures at which other proteins in the extract are degraded.

Typically, the at least one divalent cation in the solution may be Calcium and/or Magnesium. For example, the solution may contain Calcium Chloride and/or Magnesium Chloride.

Zinc, copper and manganese are also cations.

The Calcium and Magnesium ions may be included in the extract in an amount to permit control of the denaturation of limit dextrinase when the extract is heated. Typically, Calcium and Magnesium are included in the extract in an amount to at least limit the denaturation of limit dextrinase when the extract is heated. For example, the concentration of Calcium may be less than 100 mM and the concentration of Magnesium may be less than 100 mM.

A concentration of Calcium and Magnesium in a range between about 25 to 50 mM is particularly useful as further down stream processing of the extract for further purification, such as anion exchange chromatography, may require removal of Calcium and Magnesium. Accordingly a concentration of Calcium ions of about 50 mM and a concentration of Magnesium ions of about 50 mM is particularly useful.

In certain embodiments, the Calcium ions are provided in a concentration selected from the group consisting of 10 mM, 15 mM, 20 mM, 25 mM, 30 mM, 35 mM, 40 mM and 45 mM.

In certain embodiments, the Magnesium ions are provided in a concentration selected from the group consisting of 10 mM, 15 mM, 20 mM, 25 mM, 30 mM, 35 mM, 40 mM and 45 mM.

Typically the solution further includes a reducing agent for denaturing disulfide bonding. Typically the agent is L-cysteine or ascorbic acid, although in appropriate processing circumstances, other reducing agents might be used, including glutathione, 2 mercaptoethanol and dithiothrietol. Further, the solution may be buffered to about pH 7.5 using a suitable solution, such as Tris HCl. In particular, and with reference to the preceding, it has been found that by heating an extract buffered at pH 7.5 with 200 mM Tris HCl and containing divalent cations and a reducing agent such as 20 mM L-cysteine, the specific activity of the extract can be increased at least 5.14 fold (385.7 μmoles/min/mL) compared with a heat treated extract containing no reducing agent, Calcium or Magnesium (75.0 μmoles/min/mL).

Thus in certain embodiments, there is provided a process for purifying a limit dextrinase from a cell. The process includes a step of heating an extract of a cell formed from a solution having a pH of at least about 5 and including at least one divalent cation and a reducing agent, to increase the specific activity of a limit dextrinase in the extract.

Typically the reducing agent is L-cysteine. Useful concentrations of L-cysteine include concentrations from about 2 mM to 25 mM, although higher concentrations of L-cysteine are contemplated. The concentration of L-cysteine may be selected from the group consisting of 4 mM, 6 mM, 8 mM, 10 mM, 12 mM, 14 mM, 16 mM, 18 mM, 20 mM, 22 mM and 24 mM. Where the reducing agent is other the L-cysteine, such as ascorbic acid, these concentrations of ascorbic acid may also be used, although concentrations of ascorbic acid up to 50 mM can be used.

Typically the solution has a pH of at least about 5, although higher ranges to about pH 9.0 are particularly useful for enhancing the specific activity of the enzyme in the extract. A solution having a pH selected from the group consisting of 5.5, 6.0, 6.5, 7.0, 7.5, 8.0 and 8.5 may be used. A pH of above 9.0 could be used, however above this range the activity of the enzyme tends to be affected. Trizma base buffered in HCl in a concentration of about 200 mM is particularly useful to provide the appropriate pH.

It is believed that the major constituents of an extract of a barley rootlet include a number of enzymes having activity for various carbohydrate and protein substrates. Thus, the extract is typically heated to a temperature that permits denaturation of unwanted proteases, ancillary enzymes, or otherwise, destruction of activity of these enzymes in the extract. As described herein, temperatures less than 65° C. are suitable for this purpose.

It is particularly advantageous to heat the extract to between about 35 and 60° C. because at temperatures approaching 65° C. and above, limit dextrinase activity may be lost. Accordingly, a temperature of about 55° C. is particularly useful.

The inventor has also found that the purification of limit dextrinase from a barley cell extract can be improved by extracting a barley cell homogenate at 40° C. in a solution including Calcium and Magnesium. Specifically, as described herein, the specific activity of an extract comprising Calcium and Magnesium after maintenance at 40° C. was found to be 303.86-3 μmoles/min/mL as compared with the activity of an extract maintained at 40° C. in the absence of Calcium and Magnesium and L-Cysteine (65.12⁻³ μmoles/min/mL).

It is believed that maintenance of such an extract at 40° C. is important because it permits limit dextrinase to disassociate from solids in the extract, and accordingly, to solubilise into the liquid phase of the extract, prior to further processing of the extract, such as a heat treatment step or a chromatographic separation step. The Calcium and Magnesium are believed to be important for limiting hydrolysis of the enzyme during the maintenance of the extract at 40° C. Thus in accordance with the invention, a process for purifying limit dextrinase from a barley cell includes the following steps:

(a) releasing limit dextrinase from a barley cell into a solution including Calcium and Magnesium Chloride to form an extract; and

(b) heating the extract to increase the specific activity of limit dextrinase in the extract.

Typically, the extract is maintained in conditions for promoting stabilization of the limit dextrinase in the extract prior to heating the extract.

The extract may be maintained at less than 10° C. for less than 3 days. For example, the extract may be maintained between 0 to about 4° C. for between about 1 to 48 hours.

In certain embodiments, the solution further includes a reducing agent as discussed above in concentrations as discussed above. The solution is further buffered to a pH range as discussed above. Thus, in another aspect, the invention provides a process for purifying limit dextrinase from a barley cell. The process includes the following steps:

(a) releasing limit dextrinase from a barley cell into a solution having a pH of at least about 5.0, the solution including calcium, magnesium and a reducing agent, to form an extract;

(b) heating the extract to increase the specific activity of limit dextrinase in the extract.

It is particularly advantageous to maintain the extract for 12 hours at 4° C. prior to extraction at 40° C. as this improves the speed of purification protocols that comprise further purification steps.

It has further been found that limit dextrinase can be purified to virtual homogeneity from a barley cell extract by a process including the following steps:

(a) releasing limit dextrinase from a barley cell into a solution including Calcium and Magnesium and a reducing agent, the solution being at least about pH 5.0 to form an extract;

(b) heating the extract to increase the specific activity of limit dextrinase in the extract; and

(c) utilising chromatography to purify limit dextrinase from the heated extract.

As described herein, limit dextrinase can be further purified from a heat treated barley cell extract by anion exchange chromatography. Accordingly, typically, in step (c), anion exchange chromatography is utilised to purify limit dextrinase from the heated extract.

It has been found that Calcium and Magnesium ions and L-Cysteine tend to limit binding of limit dextrinase during anion exchange chromatography. Accordingly, typically the extract is desalted before anion exchange chromatography. One way of desalting to remove Calcium and Magnesium ions and L-Cysteine is by ultrafiltration. Alternatively, a preparative de-salting column, such as a Hi Prep 26/10 desalting column can be used. It is particularly advantageous to remove substantially all of the Calcium and Magnesium from the extract prior to anion exchange chromatography for the purpose of maximising the yield of limit dextrinase purified from the anion exchange column.

Typically, the extract is maintained in conditions for promoting solubilisation of the limit dextrinase in the extract prior to heating the extract.

In the processes of the invention described above, the extract of the barley cell is typically produced by homogenising barley rootlets in an appropriate buffer. One way of homogenizing grains is by use of a blender, such as a Waring blender. Alternatively, the extract may be produced by milling barley grains in an appropriate buffer using a roller mill following a predetermined steeping and germination.

As discussed above, the solution into which the limit dextrinase from the cell is released to form an extract is typically a buffer for controlling pH. Solutions prepared from Trisma base are examples of such a solution. A solution having a concentration of no more than about 300 mM Tris is suitable, for example, 200 mM Tris is particularly advantageous adjusted and maintained at a pH 7.5.

It will be understood that the processes of the invention are useful for purifying limit dextrinase from cells other than barley cells. Other examples include cells of grains such as rice and wheat, and other vegetable matter. Further, it will be understood that processes of the invention are useful for isolating barley limit dextrinase from cells that contain a recombinant nucleic acid molecule that encodes barley limit dextrinase. Examples of such cells include bacterial cells and yeast cells.

### EXAMPLE 1

### Materials and Equipment

Germinating barley seeds (Schooner variety) were obtained from Barrett Burston Malting, (Thornleigh, NSW, Australia), Calcium Chloride, Magnesium Chloride, Potassium Chloride, Sodium Chloride, Trisma base, Sodium Acetate, Hydrochloric Acid and L-Cysteine were supplied by Sigma Aldrich (Castle Hill, NSW, Australia), Red Pullulan was obtained from Megazyme (Bray, Ireland) and undenatured Ethanol was purchased from CSR Distilleries (Ingleburn, NSW, Australia).

The germinated barley grains were milled on a Kustnel Freres & Cie roller mill to a gap setting of 1 mm to crack the grains allowing extraction of enzymes.

The crude enzyme extract was coarse filtered though double cheesecloth then centrifuged at 26,800×g for 30 minutes at 4° C. to remove any precipitate.

The crude extract was concentrated and buffer exchanged using a MidGee cross flow ultrafiltration unit combining a Masterflex economy drive peristaltic pump and Masterflex Easy load II head, UFP-30-H24LA ultrafiltration cartridge with 30 kDa nominal cut off and MidGee starter kit KMDG-1. A flow rate of 17 mL per minute at 10 psi pressure was sufficient to separate and concentrate the limit dextranase containing fractions.

The buffer used for FPLC gel filtration and ion exchange chromatography was 25 mM Sodium Acetate (pH 5.5). The eluent buffer for ion exchange chromatography included 1 M NaCl.

An Amersham Pharmacia AKAT gradient processing FPLC system complete with a 900 model monitor, lamp and detector (set at 280 nm), 920 model pump and Frac 950 fraction collector interfaced to a Compaq Deskpro Pentium III computer supporting Unicorn analytical software was used for all protein purification. The columns used included a Hi Prep 26/10 desalting column connected to a Super loop 50 (to facilitate larger injection volumes), a 16/10 Hi-Prep DEAE FF anion exchange column with a final purification undertaken on a Mono Q HR 5/5 column.

Isolation of limit dextranase was identified by the presence of single protein bands on native electrophoresis gels and single absorption peaks by sequential anion exchange chromatography.

An LW Scientific UV-Visible spectrophotometer was used to measure enzyme activity operating at 510 nm. The system was controlled by a Celeron processor computer operating a LW Scientific Graphite version 3.1 enzyme kinetics software program.

### EXAMPLE 2

### Preparation of a Standard Curve for Dye Labelled Red Pullulan to Determine Limit Dextrinase Activity

A standard curve for the identification of Pullulanase activity was supplied by Megazyme utilising a pullulan substrate derived from Bacillus acidopullulyticus. The extracted pullulan is standardised for molecular weight and degree of α (1-6) branching by the action of borohydride and conjugated with Procion Red MX-5B to an extent of one dye molecule per an estimated 30 sugar residues. The Red pullulan substrate (0.5 g) is added to 25 ml of 0.5M Potassium Chloride and vortexed until completely dissolved. Working standards are prepared in the range of 100 to 800 μM/mL in 25 mM Sodium Acetate buffer at pH 5.5 and read spectrophotometrically at 510 nm. The solution is stored at 4° C. in a well sealed glass bottle with an overlay of toluene to prevent microbial infection until required. A

### EXAMPLE 3

### Preparation of a Standard Curve for Protein to Determine Limit Dextrinase Specific Activity

Protein was determined using the BioRad micro assay procedure derived from the original method of Bradford utilising a standard curve produced for bovine serum albumin. Each analysis was conducted in duplicate requiring incubation at room temperature for 10 minutes with the absorbance measured at 595 nm. Standards were prepared in the range of 0.2 to 1.4 mg/mL of protein.

### EXAMPLE 4

### Enzyme Kinetics Assay

The assay requires 1 ml of the extracted enzyme solution [suspended in 200 mM Sodium Acetate buffer at pH 5 (post buffer exchange)] pre-equilibrated at 40° C. for 5 minutes. To this suspension is added 0.5 mL Red pullulan substrate [(0.5 g) in 25 ml of 0.5M Potassium Chloride]. The mixture is stirred and incubated at 40° C. for exactly 10 minutes. The reaction is terminated by the addition of 2.5 ml of 95% (v/v) ethanol vortexing for 10 seconds. The reaction tubes are allowed to equilibrate at room temperature for 10 minutes and then centrifuged at 1,000×g to precipitate the higher molecular weight fractions of the substrate. The supernatant is transferred directly to a curvette and the absorbance read at 510 nm. Activity is determined by reference to the standard curve. A reference blank is prepared by adding 1 mL of distilled water to 2.5 mL ethanol and 0.5 mL of the Red Pullulan substrate.

### EXAMPLE 6

### Preparation of Crude Limit Dextrinase Extract

30 g of 3 to 12 month old stored barley grains were dispersed in 45 mL 0.2M Tris-HCl (pH7.5) containing 20 mM L-Cysteine, 50 mM MgCl₂ and 50 mM CaCl₂ following a germination period. The germinated grains were firstly milled using smooth rollers at a gap setting of 1 mm and speed 440 rpm, feed rate of 1 kg per minute prior to extraction at 40° C. for 2 hours to facilitate solubilisation of limit dextrinase.

The insoluble material was removed from the extract by filtering through double cheese cloth. The filtrate was centrifuged at 15,000 rpm for 30 minutes at 4° C. to remove solids and the supernatant was passed through a 0.45 μM filter and stored at 4° C. in a sterile container with 0.01% sodium azide. This process formed the crude limit dextrinase extract.

The activity and specific activity of the crude limit dextrinase extract was then determined according to Examples 2, 3 and 4 above.

### EXAMPLE 7

### Purification of Limit Dextrinase from the Crude Limit Dextrinase Extract

The first stage of the purification process involved the removal of heat labile proteases, inhibitory proteins and any superfluous proteinaceous materials from the crude limit dextrinase extract with the aim of reducing any loss of activity or damage to the structure of limit dextrinase while increasing the specific activity of limit dextranase extract. To inactivate and remove these proteins, the crude extract was heated in a water bath to 55° C. and maintained at that temperature for 1 hour. The extract was then cooled to room temperature and buffer exchanged by cross flow ultrafiltration with 25 mM Sodium Acetate pH 5.5 to facilitate gel filtration and ion exchange chromatography. The extract was initially centrifuged and filtered through a 0.45 μm filter.

The activity and specific activity of the heat treated limit dextrinase extract was then determined according to Examples 2,3 and 4 above.

Gel filtration and ion exchange chromatography was then undertaken. A 50 mL sample of the extract was injected into a Super loop 50 column and gel filtered by FPLC on a Hi Prep 26/10 desalting column at a flow rate of 7.0 mL per minute, to remove magnesium, calcium and L-Cysteine. The desalted fractions were then pooled and reloaded onto the Super loop column and passed through a Hi PREP 16/10 DEAE anion exchange column at 3.0 mL per minute to initially fractionate limit dextrinase. The isolated fraction was again desalted to remove the 1M NaCl elution buffer and purified by passing the fraction through the Mono Q HR 5/5 column at 1.5 mL per minute. A single peak was obtained and analysed for activity and specific activity according to Examples 2 to 4 above.

### EXAMPLE 8

### Purification Profile for Limit Dextrinase

The results for the purification of limit dextrinase are shown in Table 1.

TABLE 1

| | | Specific | Purification |
|---|---|---|---|
| Sample | Activity | activity | factor |
| Crude extract | 411.96 | 312.09 | 1 |
| Heat treatment | 416.1 | 756.54 | 2.42 |
| Anion exchange | 420.05 | 8738.52 | 28 |

Activity: ×10³ μmoles/min/mL
Specific activity: ×10³ μmoles/min/mL/mg

### EXAMPLE 9

### Effect of Calcium. Magnesium and L-Cysteine on Limit Dextranase Activity of Crude Extract During Solubilization at 40° C.

We sought to determine whether calcium magnesium and L-Cysteine would have an effect on stabilisation of limit dextrinase in the crude extract, or otherwise, on preserving or enhancing limit dextrinase activity of the crude extract, during the step of extracting limit dextrinase at 40° C. that follows the milling step described in Example 6.

To this end we extracted the enzyme in (i) water, (ii) 0.2M Tris-HCL (pH 7.5), (iii) 0.2M Tris-HCL (pH 7.5) with 50 mm Calcium Chloride and 50 mm Magnesium Chloride, (iv) 0.2M Tris-HCL (pH 7.5) with 50 mm Calcium Chloride and 50 mm Magnesium Chloride and 20 mM L-Cysteine and maintained the extract at 40° C. for 2 hours. We found that the buffer containing 0.2M Tris-HCL maintained at a pH of 7.5 with the addition of 50 mM Calcium Chloride and 50 mM Magnesium Chloride in the presence of 20 mM L-Cysteine, enhanced and indeed stabilised limit dextranase activity compared to water (observed over a decreasing range of pH). The limit dextranase activity was 466% greater than in the sample with no Calcium, Magnesium or L-Cysteine at decreasing pH, (303.86μmoles/min/mL compared to 65.12 μmoles/min/mL).

### EXAMPLE 10

### Effect of Calcium, Magnesium L-Cysteine on Limit Dextrinase Activity Maintained at pH 7.5 of Crude Extract During Heat Treatment

We sought to determine whether calcium, magnesium and L-Cysteine would have an effect on stabilisation of limit dextrinase in the crude extract or otherwise on preserving or enhancing limit dextrinase activity of the crude extract, during the step of heating limit dextrinase as described in Example 7.

To this end, we incubated the crude extract at temperatures from 25° C. to 65° C. for 1 hour in 0.2M Tris-HCL (pH 7.5) with 50 mm Calcium Chloride and 50 mm Magnesium Chloride and 20 mM L-Cysteine We found that after heating, the activity dropped significantly in samples held above 65° C. (13.6 μmoles/min/mL) compared to extracts held at 25° C. (168.16 μmoles/min/mL), 40° C. (251.47 μmoles/min/mL) and 55° C. (242.68 μmoles/min/mL) and as a consequence consolidated the extraction process to 40° C. and heating to 55° C.

1. A process for purifying a limit dextrinase from a cell including the step of heating an extract of a cell formed from a solution including at least one divalent cation, to increase the specific activity of a limit dextrinase in the extract.

2. The process according to claim 1 wherein the divalent cation is calcium or magnesium.

3. The process according to claim 1 wherein the divalent cation is calcium and magnesium.

4. The process according to claim 1 wherein the divalent cation is provided in the solution in a concentration of less than about 100 mM.

5. The process according to claim 1 wherein the solution further includes a reducing agent for denaturing disulfide bonding.

6. The process according to claim 5 wherein the reducing agent is L-cysteine.

7. The process according to claim 6 wherein the concentration of L-cysteine in the solution is less than about 25 mM.

8. The process according to claim 1 wherein the extract has a pH of at least about 5.

9. The process according to claim 1 wherein the solution is heated to less than about 65° C.

10. A process according to claim 1 wherein the cell is a barley cell.
